(19) United States
(12) Patent Application Publication
(10) Pub. No.: US 2019/0336677 A1
(43) Pub. Date: Nov. 7, 2019

(54) SUCTION AND IRRIGATION APPARATUS

(71) Applicant: Multigate Medical Products Pty Ltd, Villawood (AU)

(72) Inventors: Mark Spaile, Villawood (AU); Shirley Liu, Australia (AU)

(73) Assignee: Multigate Medical Products Pty Ltd, Villawood, AU (unknown)

(21) Appl. No.: 16/312,854

(22) PCT Filed: Jun. 6, 2017
(86) PCT No.: PCT/AU2017/050556

§ 371 Date: Dec. 21, 2018

(30) Foreign Application Priority Data
Jun. 24, 2016 (AU) 2016902489

(51) Int. Cl. A61M 3/02

(52) U.S. Cl. CPC A61M 3/0283; A61M 3/022

(57) ABSTRACT

The invention is directed to an apparatus for providing suction and irrigation to a body site. The apparatus includes a shaft having a distal end for juxtaposition with the body site, and an actuating mechanism operatively configured to facilitate selective actuation of the apparatus to provide either suction or irrigation via the shaft. The shaft has a flow area restriction portion located proximate its distal end.

### TECHNICAL FIELD

The invention described herein relates to a suction and irrigation apparatus. In particular, embodiments of the invention is directed to a suction and irrigation apparatus for use in medical procedures.

### BACKGROUND ART

During certain medical procedures, such as surgery or dental procedures, it is often necessary for a medical professional to provide irrigation fluid to a body site such as a wound or oral cavity. The irrigation fluid may be, for example, water, saline, or another biocompatible fluid. At the same time, it is often necessary to also apply suction to the body site in order to remove fluid and debris.

For example, during keyhole surgery, such as keyhole surgery for the abdomen, it is often necessary to remove debris by suction, and to irrigate the body site by delivering an irrigation fluid. The suction can also be used to vent smoke created by cauterisation during surgery. In keyhole surgery procedures for the abdomen, the abdomen is typically pumped up by gas to provide improved visibility and working space. To avoid collapsing the abdomen when venting the gas, it is critical to ensure that a small amount of gas is vented in a controlled manner. In addition, the medical professional must ensure that suction forces are not overly strong so as to avoid tissue damage. When providing irrigation to the body site, it is also desirable to provide the irrigation fluid in a focused and accurate manner.

Embodiments of the present invention provide a suction and irrigation apparatus which assists a medical professional to provide suction and irrigation in a controlled manner during surgical procedures where a high level of precision is required.

### SUMMARY OF THE INVENTION

According to one aspect of the invention, there is provided an apparatus for providing suction and irrigation to a body site, the apparatus including

a shaft having a distal end for juxtaposition with the body site, and

an actuating mechanism operatively configured to facilitate selective actuation of the apparatus to provide either suction or irrigation via the shaft,

wherein the shaft has a flow area restriction portion located proximate its distal end.

Typically, the flow area restriction portion is slightly offset from the distal end.

Advantageously, the flow area restriction portion enables the flow of irrigation fluid proximate the shaft distal end to become focused so that the irrigation fluid can be delivered in a precise and controlled manner during a medical procedure.

The flow area restriction portion may be of any suitable shape and size to provide a reduction in the effective flow area for the irrigation fluid proximate the distal end of the shaft. The flow area restriction portion may provide a section of constant reduced cross-sectional flow area proximate the distal end of the shaft.

In one embodiment, the flow area restriction portion may include an insert located inside the shaft, the insert having a cross-sectional flow area smaller than that of the shaft. A space may be defined between a side wall of the shaft and a side wall of the insert.

In another embodiment, the flow area restriction portion may include an integral neck portion of the shaft, the neck portion providing a narrowed cross-sectional flow area.

Providing an area proximate the distal end of the shaft where the cross-sectional flow area is reduced increases the velocity of the irrigation fluid as it flows through the flow area restriction portion. In this manner, the velocity of the irrigation fluid is increased slightly as it approaches the open end of the shaft, allowing the irrigation fluid to exit the shaft in a more focused and controlled manner.

The shaft may have a cylindrical side wall defining one or more apertures proximate its distal end. Advantageously, when the apparatus is providing suction functions, the apertures reduce the negative pressure and thus suction forces delivered to the body site so as to avoid tissue damage and collapsing the abdomen during keyhole surgery.

Any suitable number of apertures may be provided. In one embodiment, the side wall of the shaft defines two rows of apertures proximate the distal end. Preferably, eight evenly spaced apart apertures may be provided proximate the distal end of the shaft.

Preferably, the apertures are generally located between the flow area restriction portion and the distal end of the shaft. When providing irrigation, irrigation fluid flows through the shaft. As the flow area restriction portion provides a reduced cross-sectional flow area, velocity of the irrigation fluid increases as it passes the reduced cross-sectional flow area of the flow area restriction portion. Such an increase in flow velocity of the irrigation fluid allows the irrigation fluid to flow past the apertures and through an opening at the distal end of the shaft. In this manner, the flow of the irrigation fluid is more focused and the fluid can be delivered in a precise and controlled manner to a specific body site.

However, in another embodiment, where the flow restriction area is provided by an insert, the insert may cover some or all of the apertures. In this embodiment, it is preferred that a space is provided between an outer wall of the insert and the apertures, so that relief from tissue damage is available during suction.

The apparatus may include a body providing a handle portion to allow a user to hold and operate the apparatus. The body may include one or more terminals for mounting the shaft. A proximal end of the shaft may be mounted to a terminal of the body.

The apparatus may further include an adaptor for mounting two or more conduits to a terminal of the body, at least one conduit being an irrigation conduit for delivering irrigation fluid to the terminal, and at least one conduit being a suction conduit for providing a negative pressure at the terminal. The adaptor may provide fluid communication between the shaft and the irrigation conduit, as well as the shaft and the suction conduit, when the shaft is mounted to the terminal.

The body may have two terminals for selectively receiving the proximal end of the shaft. The two terminals may be disposed generally perpendicularly to one another. A user can therefore selectively mount the shaft to either one of the two terminals to suit user preferences and different applications. The apparatus may include an internal conduit for connecting, and providing fluid communication between, the two terminals. One end of the internal conduit may be mounted to the adaptor for fluid communication with the suction conduit and the irrigation conduit.

The actuation mechanism may be used to selectively open or close the suction conduit or irrigation conduit so as to provide either suction or irrigation functions via the shaft.

The actuation mechanism may include a suction button for actuating suction functions of the apparatus, and an irrigation button for actuating irrigation functions of the apparatus. In one embodiment, depression of the suction button may cause the suction conduit to be opened and irrigation conduit to be closed. Similarly, depression of the irrigation button may cause the irrigation conduit to be opened and suction conduit to be closed.

The actuation mechanism may allow the degree and pressure of suction and irrigation to be controlled by controlling the degree of depression of each of the suction and depression buttons. For example, the amount of suction or irrigation delivered to the body site may be reduced by reducing the degree of depression of the suction or irrigation button respectively. In the event that the apparatus is used for venting smoke, a gentle suction action is typically required at the body site. To provide gentle suction, the suction button can be pressed gently by a user such that the suction button is not depressed to its full extent. Similarly, the irrigation button can be pressed gently to deliver gentle irrigation to the body site.

Each button may be spring loaded and biased in a fully extended position. In the fully extended position, a protrusion associated with each button in combination with internal projections of the body may compress a corresponding conduit into a closed condition. In this manner, when both the suction and irrigation buttons are released and biased in the fully extended positions, both the suction and irrigation conduits are clamped closed.

According to another aspect of the invention, there is provided a shaft for a suction and irrigation apparatus, the shaft having

a distal end for juxtaposition with a body site, and

a flow restricting portion located proximate its distal end.

In order that the invention may be more readily understood and put into practice, one or more preferred embodiments thereof will now be described, by way of example only, with reference to the accompanying drawings.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristic described herein may be combined in any suitable manner in one or more combinations.

### BRIEF DESCRIPTION OF THE DRAWINGS

FIG. 1A is a perspective view of a partially assembled apparatus for providing suction and irrigation to a body site according to one embodiment of the invention (showing internal components).

FIG. 1B is a side view of the apparatus illustrated in FIG. 1A.

FIG. 1C is a side view of the apparatus of FIGS. 1A and 1B in its completely assembled form.

FIG. 2A is a perspective of the partially assembled apparatus illustrated in FIGS. 1A and 1B when the apparatus is configured to operate in a different orientation.

FIG. 2B is a side view of the apparatus illustrated in FIG. 2A.

FIG. 2C is a side view of the apparatus of FIGS. 2A and 2B in its completely assembled form.

FIG. 3 is an exploded assembly view of a body of the apparatus illustrated in FIGS. 1A to 2C.

FIG. 4A is a side view of a shaft of the apparatus according to one embodiment of the invention.

FIG. 4B is a cross sectional view of a distal end of the shaft illustrated in FIG. 4A.

FIG. 4C is a cross sectional view of a distal end of the shaft as illustrated in FIG. 4B in which the insert is omitted.

FIG. 4D is a cross sectional view of a distal end of the shaft in which the insert partially covers the apertures according to one embodiment of the invention.

FIG. 4E is a cross sectional view of a distal end of the shaft in which the insert completely covers the apertures according to one embodiment of the invention.

FIG. 5A is a perspective view of a shaft of the apparatus according to another embodiment of the invention.

FIG. 5B is a cross sectional view of a distal end of the shaft illustrated in FIG. 5A.

### DETAILED DESCRIPTION OF PREFERRED EMBODIMENT(S)

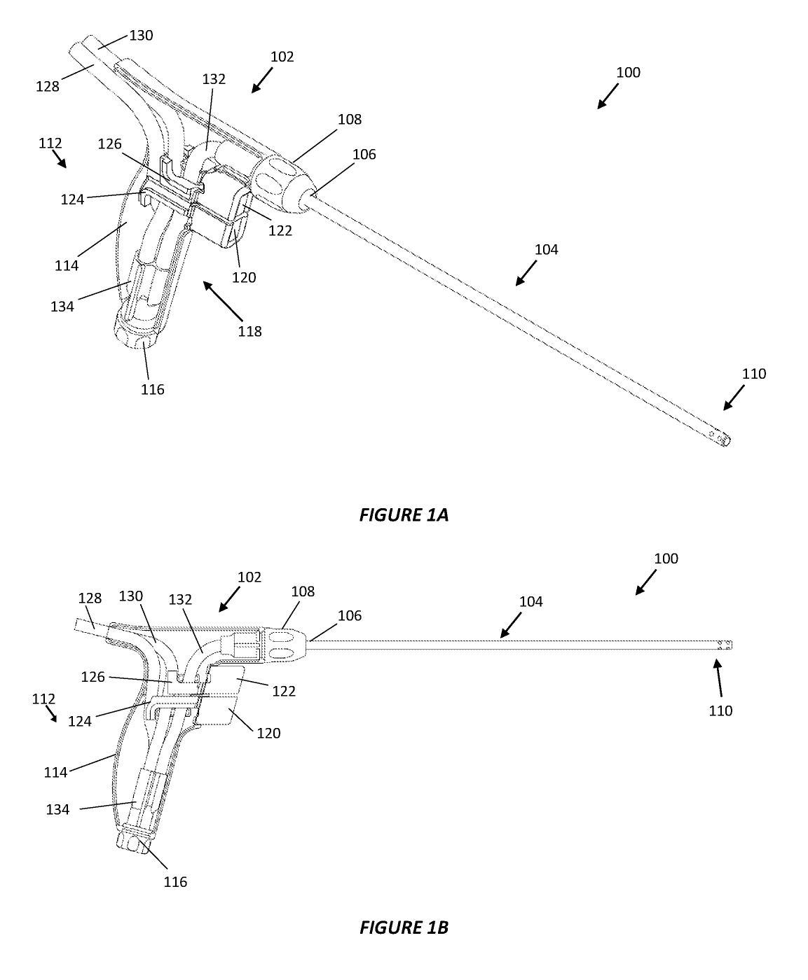

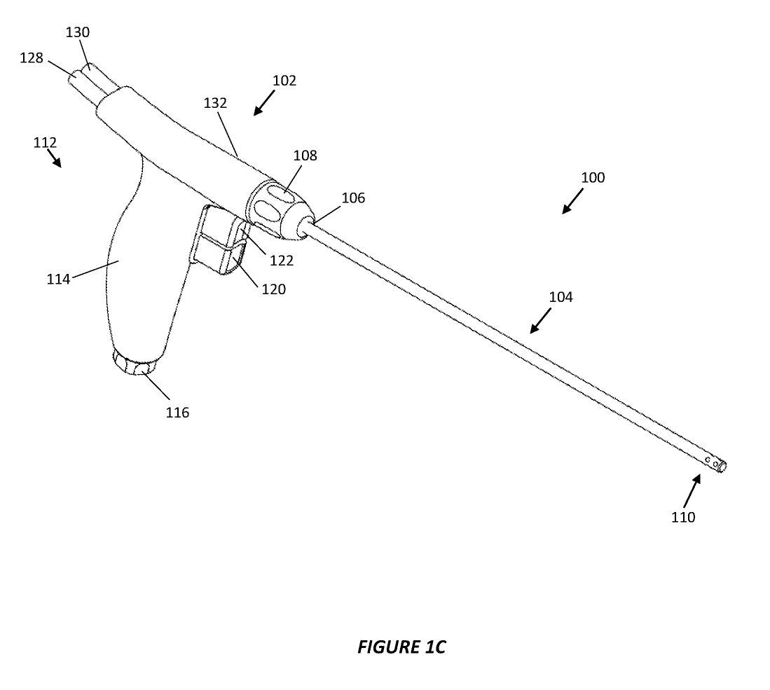

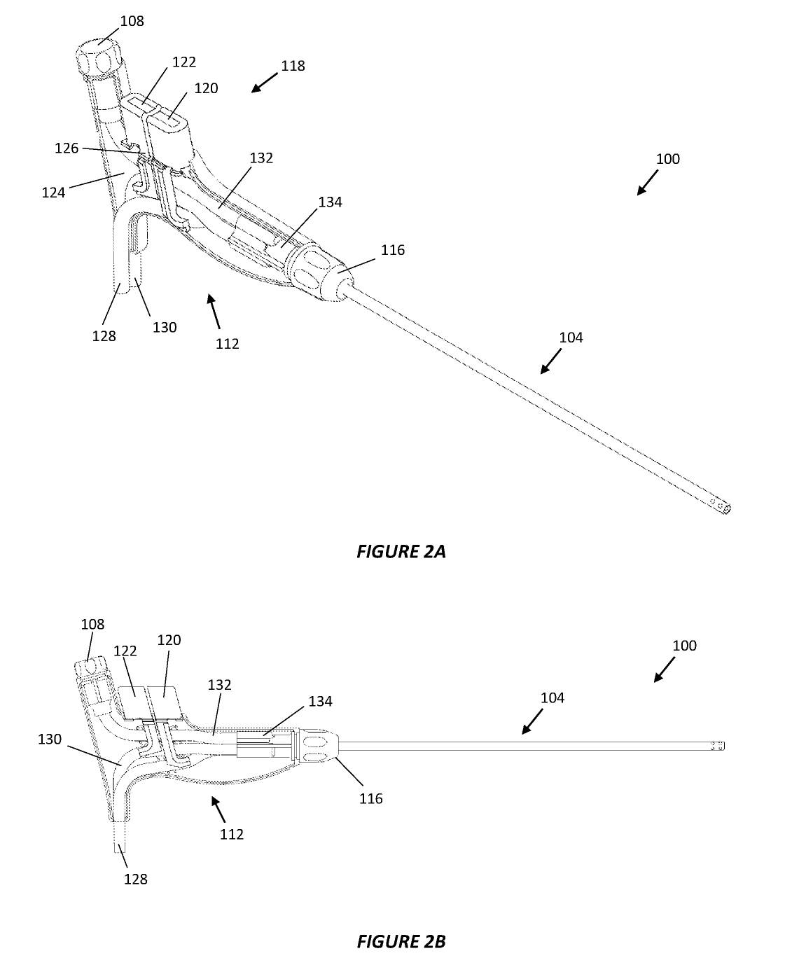

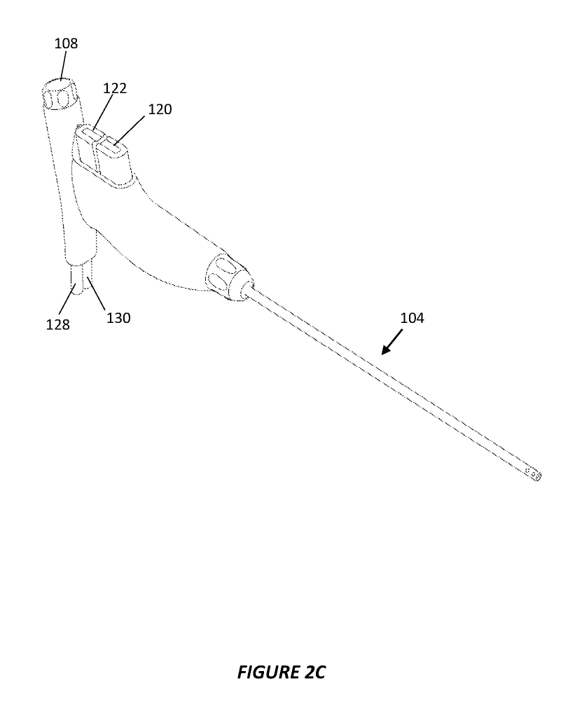

As shown in FIGS. 1A to 2C, an apparatus 100 for providing suction and irrigation to a body site (not shown) includes a body 102 and a shaft 104 attached thereto. The shaft 104 has a proximal end 106 for mounting to a first terminal 108 of the body 103, and a distal end 110 for juxtaposition with the body site.

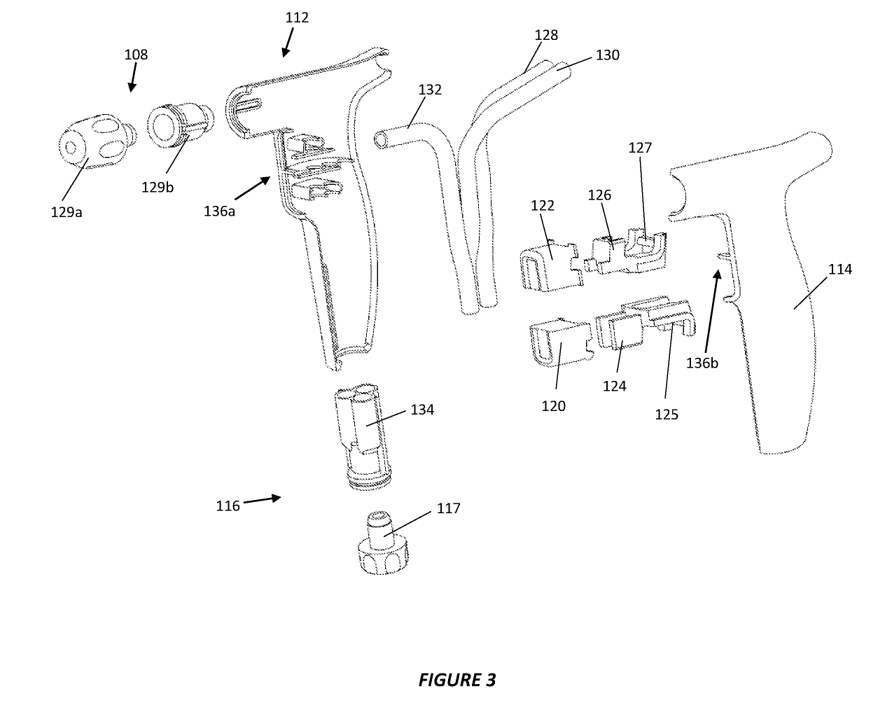

The body 102 includes a housing 112 for covering various internal components of the apparatus 100 as described in further detail below with reference to FIG. 3. The housing 112 includes a handle portion 114 to facilitate a user to hold and operate the apparatus 100. The body 102 includes a first terminal 108 and a second terminal 116 for selectively receiving the proximal end 106 of the shaft 104. The first terminal 108 is disposed generally perpendicularly to the second terminal 116.

As more clearly shown in FIG. 3, the apparatus 100 further includes an irrigation conduit 128 for delivering irrigation fluid to the shaft 104, and a suction conduit 130 for providing a negative pressure at the shaft 104 so that suction can be carried out at the body site. The apparatus 100 further includes an internal conduit 132 for connecting, and providing fluid communication between, the first and second terminals 108, 116. When fully assembled, the conduits 128, 130, 132 are held in place by internal projections 136a, 136b of the housing 112.

When the shaft 104 is mounted to the first terminal 108, connectors 129a, 129b at the first terminal 108 provide sealing fluid communication between the shaft 104 and the internal conduit 132. In the operating orientation as shown in FIGS. 1A to 1C, the second terminal 116 is closed, and the adaptor 134 provides sealing fluid communication between the internal conduit 132 and the suction conduit 130, as well as the internal conduit 132 and the irrigation conduit 128.

Now referring to FIGS. 1A, 1B and 3, the apparatus 100 includes an actuating mechanism 118 operatively configured to facilitate selective actuation of the apparatus 100 to provide either suction or irrigation via the shaft 104. The actuating mechanism 118 includes a suction button 120, and an irrigation button 122. Each button 120, 122 is associated with an extension 124, 126 such that, upon depression of a respective button 120, 122, the associated respective extension 124, 126 is pushed rearward to compress a respective suction or irrigation conduit 128, 130 against a portion of the internal projections 136a, 136b into a closed condition so that the appropriate suction or irrigation function is provided via the shaft 104. Each button 120, 122 is spring loaded (not shown) and biased in protruded fully extended position as shown in FIGS. 1A to 1C so that each button 120, 122 automatically returns to its fully extended position once a user is no longer holding the respective button 120, 122 in the depressed position.

For example, as more clearly shown in FIG. 1B, if irrigation button 122 is depressed and the suction button 120 is not depressed, extension 126 is pushed rearwardly to compress suction conduit 130 against the internal projections 136a, 136b into a closed condition, whilst the irrigation conduit 128 remains open. Therefore, when the irrigation button 122 is depressed, irrigation fluid can be delivered to the shaft 104 via the open irrigation conduit 128 and the internal conduit 132, and suction is blocked by compression of the suction conduit 130.

Similarly, when the suction button 120 is depressed, and the irrigation button 122 is released, extension 124 is pushed rearwardly to compress irrigation conduit 128 against the internal projections 136a, 136b into a closed condition, whilst the suction conduit 130 remains open. Therefore, when the suction button 120 is depressed, a negative pressure can be delivered to the shaft 104 via the open suction conduit 130 and the internal conduit 132 to provide suction to the body site, and irrigation is blocked by compression of the irrigation conduit 128.

When the suction and irrigation buttons 120, 122 are released and biased in their fully extended positions, a rear portion of each of the associated extensions 125, 127 pushes against the internal projections 136a, 136b so that both conduits 128, 130 are clamped in a closed condition. In this manner, when both buttons 120, 122 are released by a user, the apparatus 100 is turned off.

Now referring to FIGS. 2A to 2C, these illustrate use of the apparatus 100 in a different orientation. In particular, shaft 104 is mounted to the second terminal 116 of the body 112, bypassing internal conduit 132. When operating in this orientation, the first terminal 108 is closed and adaptor 134 associated with the second terminal 116 provides sealing fluid communication between the shaft 104 and the irrigation conduit 128, as well as the shaft 104 and suction conduit 130. Accordingly, operation of the actuation mechanism 118 as previously described with reference to FIGS. 1A, 1B and 3 selectively provides suction or irrigation via the shaft 104.

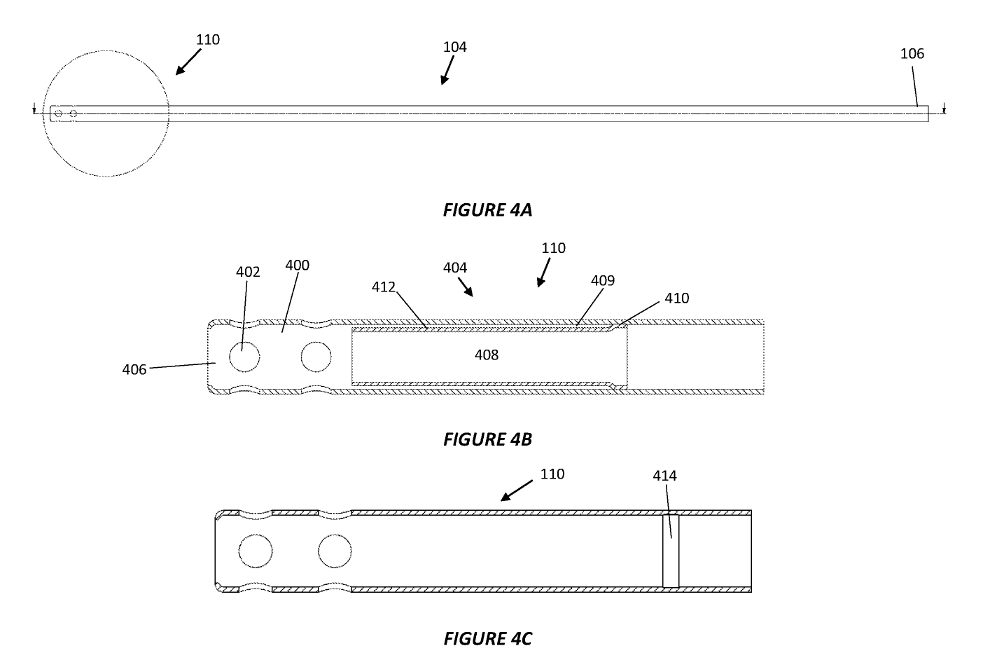

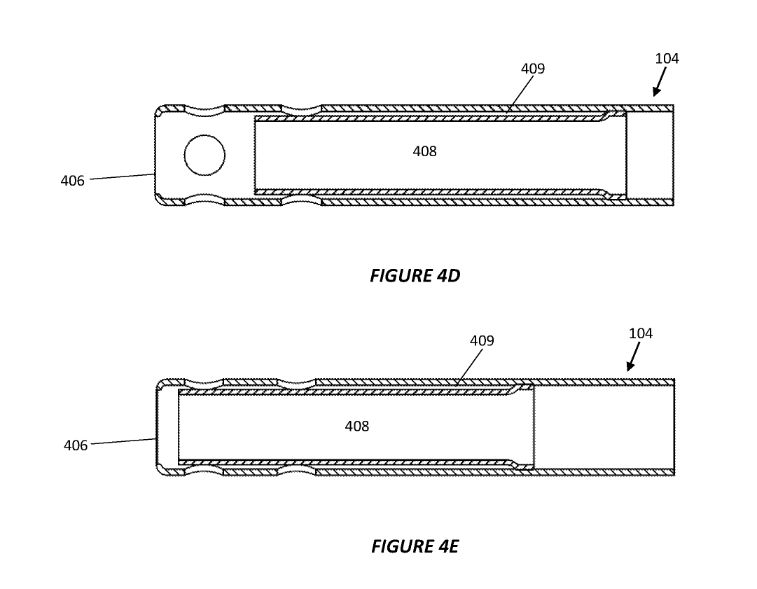

A shaft 104 according to one embodiment of the invention is shown more clearly in FIGS. 4A to 4C. As previously described, the shaft 104 has a proximal end 106 of the shaft is mounted to the body 102 of the apparatus 100, and a distal end 110 delivers irrigation or suction to a body site.

As more clearly shown in FIG. 4B, a cylindrical side wall 400 of the shaft 104 defines a plurality of apertures 402 proximate its distal end 110. In particular, the side wall 400 defines eight evenly spaced apertures 402 arranged in two rows. The apertures 402 slightly reduce the force created during suction at the distal end 110 of the annular 104 so as to provide gentle suction to the body site in an accurate and controlled manner, thereby avoiding tissue damage, and collapsing the abdomen during keyhole surgery.

However, when providing irrigation, irrigation fluid can flow through the apertures 402 undesirably causing the delivery of the irrigation fluid to become unfocused and imprecise. A flow area restriction portion 404 is provided proximate the distal end 110 of the shaft 104 to increase the velocity of the irrigation fluid flowing through the distal end 110 such that the irrigation fluid flows past the apertures and exits the shaft 104 through end opening 406 in a more focused and controlled manner. The flow area restriction portion 404 thereby provides increased accuracy in the delivery of irrigation fluid to the body site, which can be critical in precision surgery procedures.

In the embodiment shown in FIG. 4B, the flow area restriction portion 404 includes a sleeve insert 408 located inside the shaft 104 adjacent the apertures 402. The sleeve insert 408 includes an enlarged end portion 410 for abutment with an inner wall of the shaft 104. As shown in FIG. 4C, a groove 414 is provided on the inner wall of the shaft 104 to hold the sleeve insert 408 in position, by engaging with enlarged end portion 410. The insert 408 includes a narrowed portion 412 adjacent the enlarged end portion 410 having an internal diameter smaller than that of the shaft 104 for providing a section of reduced flow area.

The internal diameter of the sleeve insert 408 may be any suitable size to provide the required performance characteristics for the apparatus. Typically, the internal diameter of the sleeve insert 408 is approximately 10% to 15% smaller than the internal diameter of the shaft 104. In one embodiment, the internal diameter of the shaft 104 is roughly 4.5 mm and the internal diameter of the inert is roughly 3.5 mm.

In the embodiment shown in FIG. 4B, the insert 408 is positioned adjacent the apertures 402 such that none of the apertures 402 is covered by the narrowed portion 410 of the insert. In some embodiments, as more clearly illustrated in FIG. 4D, the insert 408 can be positioned closer to the end opening 406 such that a portion of the apertures 402 are covered or partially covered by the insert 408. In other embodiments, as more clearly illustrated in FIG. 4E, the insert 408 can be positioned such that all of the apertures 402 are covered by the insert 408. Spacing 409 between the insert 408 and the inner wall of the shaft 104 allows gas to flow through the covered or partially covered apertures 402 during suction. By partially covering the apertures 402 using the insert 408 in this manner effectively reduces the overall suction rate to a desired level, whilst delivering sufficient suction to the body site to remove smoke, fluid and/or debris.

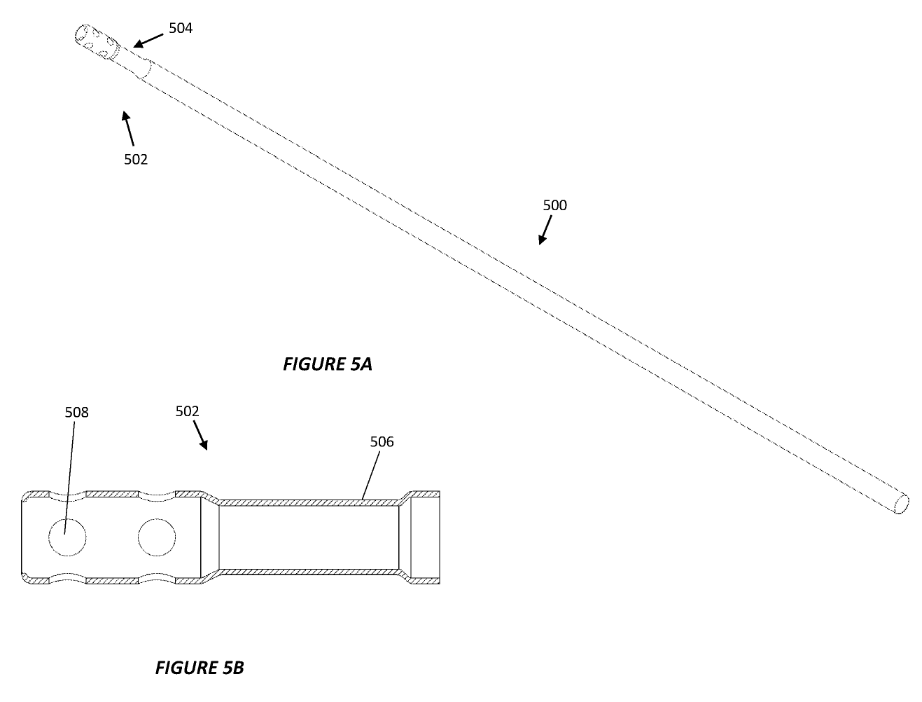

A shaft 500 according to another embodiment of the invention is shown in FIGS. 5A and 5B. The shaft 500 also includes a flow area restriction portion 502 proximal its distal end 504. The flow area restriction portion 502 includes an integral neck portion 506 having a reduced internal diameter compared to that of the shaft 500 (FIG. 5B). The internal neck portion 506 is located adjacent the apertures 508 and provides a reduction in the flow area to thereby increase flow velocity for the irrigation fluid in the same manner and operating principles as the sleeve insert 408 of FIG. 4B.

The internal diameter of the neck portion 506 may be any suitable size to provide the required performance characteristics for the apparatus 100. Typically, the internal diameter of the neck portion 506 is approximately 10% to 15% smaller than the internal diameter of the shaft 104. In one embodiment, the internal diameter of the shaft 104 is roughly 4.5 mm and the internal diameter of the neck portion 506 is roughly 3.5 mm.

Advantageously, having a flow area restriction portion proximate and slightly offset from the distal end 406 of the shaft 104 in combination with the apertures 402 increases the velocity of the irrigation stream before a pressure loss is created due to the apertures 402. This increased velocity improves the quality of the flow exiting the shaft 104. On the other hand, the suction rate is preserved by the apertures 402 so that larger particles can be removed via suction functions of the apparatus 100. At the same time, the suction rate is controlled and not excessive so as to prevent tissue damage.

In some other embodiments, the cylindrical side wall 400 of the shaft 104 and/or an internal wall of the neck portion 506 or insert 408 may be uneven or may include projections and recesses in some or all areas to reduce the effective cross sectional flow area of the shaft 104. In these embodiments, the flow quality delivered by the shaft 104 may be more turbulent than other embodiments.

In some embodiments, the apparatus 100 may have more than one flow area restriction portion along its length.

In some embodiments, the flow area restriction portion may include an area of the shaft 104 in which the cross sectional shape changes from a generally circular shape to an elliptical shape, or another shape having a reduced cross sectional flow area. Similarly, the cross sectional shape of the integral neck portion 506 and the insert 408 may have an elliptical, square, rectangular or any suitably shaped cross section.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

The term “comprise” and variants of that term such as “comprises” or “comprising” are used herein to denote the inclusion of a stated integer or integers but not to exclude any other integer or any other integers, unless in the context or usage an exclusive interpretation of the term is required.

Reference to prior art disclosures in this specification is not an admission that the disclosures constitute common general knowledge.

The claims defining the invention are as follows:

1. An apparatus for providing suction and irrigation to a body site, the apparatus including
a shaft having a distal end for juxtaposition with the body site, and
an actuating mechanism operatively configured to facilitate selective actuation of the apparatus to provide either suction or irrigation via the shaft,
wherein the shaft has a flow area restriction portion located proximate its distal end.

2. The apparatus of claim 1, wherein the flow area restriction portion is slightly offset from the distal end.

3. An apparatus according to any one of the preceding claims, wherein the flow area restriction portion provides a section of constant reduced cross-sectional flow area proximate the distal end of the shaft.

4. An apparatus according to any one of the preceding claims, wherein the flow area restriction portion includes an insert located inside the shaft, the insert having a cross-sectional flow area smaller than that of the shaft.

5. The apparatus of claim 4, wherein a space is defined between a side wall of the shaft and a side wall of the insert.

6. An apparatus according to any one of claims 1 to 3, wherein the flow area restriction portion may include an integral neck portion of the shaft, the neck portion providing a narrowed cross-sectional flow area.

7. An apparatus according to any one of the preceding claims, wherein the shaft has a cylindrical side wall defining one or more apertures proximate its distal end.

8. The apparatus of claim 7, wherein the side wall of the shaft defines two rows of apertures proximate the distal end.

9. The apparatus of claim 7 or 8, wherein eight evenly spaced apart apertures are provided proximate the distal end of the shaft.

10. An apparatus according to any one of claims 7 to 9, the apertures are generally located between the flow area restriction portion and the distal end of the shaft.

11. The apparatus of claim 4 or 5, wherein the shaft has a cylindrical side wall defining one or more apertures proximate its distal end, and the insert covers some or all of the apertures.

12. The apparatus of claim 11, wherein a space is provided between an outer wall of the insert and the apertures.

13. An apparatus according to any one of the preceding claims, wherein the body includes a terminal for mounting the shaft, and the apparatus further includes an adaptor for mounting two or more conduits to the terminal, at least one conduit being an irrigation conduit for delivering irrigation fluid to the terminal, and at least one conduit being a suction conduit for providing a negative pressure at the terminal.

14. The apparatus of claim 13, wherein the adaptor provides fluid communication between the shaft and the irrigation conduit, as well as the shaft and the suction conduit, when the shaft is mounted to the terminal.

15. The apparatus of claim 13 or 14, wherein the body has two terminals for selectively receiving a proximal end of the shaft, the apparatus further including an internal conduit for connecting, and providing fluid communication between the two terminals, one end of the internal conduit being mounted to the adaptor for fluid communication with the suction conduit and the irrigation conduit.

16. The apparatus of claim 15, wherein the two terminals are disposed generally perpendicularly to one another.

17. An apparatus according to any one of claims 13 to 16, wherein the actuation mechanism is used to selectively open or close the suction conduit or irrigation conduit so as to provide either suction or irrigation functions via the shaft.

18. The apparatus of claim 17, wherein the actuation mechanism includes a suction button for actuating suction functions of the apparatus, and an irrigation button for actuating irrigation functions of the apparatus, and wherein
depression of the suction button causes the suction conduit to be opened and irrigation conduit to be closed, and depression of the irrigation button causes the irrigation conduit to be opened and suction conduit to be closed.

19. The apparatus of claim 18, wherein the actuation mechanism allows the degree and pressure of suction and irrigation to be controlled by controlling the degree of depression of each of the suction and depression buttons.

20. The apparatus of claim 18 or 19, wherein each button is spring loaded and biased in a fully extended position.

21. The apparatus of claim 20, wherein in the fully extended position, a protrusion associated with each button in combination with internal projections of the body compresses a corresponding conduit into a closed condition, such that when both the suction and irrigation buttons are released and biased in the fully extended positions, both the suction and irrigation conduits are clamped closed.

22. A shaft for a suction and irrigation apparatus, the shaft having
a distal end for juxtaposition with a body site, and
a flow restricting portion located proximate its distal end.
